(19) United States
(12) Patent Application Publication
(10) Pub. No.: US 2025/0082507 A1
(43) Pub. Date: Mar. 13, 2025

(54) ELASTOMERIC PLUG FOR PATULOUS EUSTACHIAN TUBE

(71) Applicant: Acclarent, Inc., Irvine, CA (US)

(72) Inventors: Raymond Yue-sing TANG, Rosemead, CA (US); Henry F. SALAZAR, Pico Rivera, CA (US); Cesar FUENTES-ORTEGA, Pasadena, CA (US)

(21) Appl. No.: 18/826,094

(22) Filed: Sep. 5, 2024

Related U.S. Application Data
(60) Provisional application No. 63/536,964, filed on Sep. 7, 2023.

(51) Int. Cl. A61F 11/20

(52) U.S. Cl. CPC A61F 11/202; A61F 2230/0093

(57) ABSTRACT

The present application discloses an implant with a shaft defining a longitudinal axis and an extendable sleeve including a proximal end and a distal end. The extendable sleeve is positioned around the shaft and is configured to bear radially outward against a Eustachian tube of a patient. The distal end of the extendable sleeve is configured to translate with the shaft. The proximal end of the sleeve is configured to translate relative to the shaft. The present application also discloses an apparatus that includes the implant and an instrument that facilitates the implant between an insertion position and a released position. Finally, the present application discloses methods for use of the implant and the apparatus.

### CROSS-REFERENCE TO RELATED APPLICATIONS

This application claims the benefit of U.S. Provisional Application No. 63/536,964, filed Sep. 7, 2023, the entirety of which is incorporated herein by reference.

### BACKGROUND

Referring to FIG. 1, the ear 10 is divided into three parts: an external ear 12, a middle ear 14 and an inner ear 16. The external ear 12 consists of an auricle 18 and ear canal 20 that gather sound and direct it toward a tympanic membrane 22 (also referred to as the eardrum) located at an inner end 24 of the ear canal 20. The middle ear 14 lies between the external and inner ears 12, 16 and is connected to the back of the throat by a Eustachian tube (ET) 26, which serves as a pressure equalizing valve between the ear 10 and the sinuses. The ET 26 terminates in a pharyngeal ostium 28 in the nasopharynx region 30 of the throat 32. In addition to the eardrum 22, the middle ear 14 also consists of three small ear bones (ossicles): the malleus 34 (hammer), incus 36 (anvil) and stapes 38 (stirrup). These bones 34, 36, 38 transmit sound vibrations to the inner ear 16 and thereby act as a transformer, converting sound vibrations in the canal 20 of the external ear 12 into fluid waves in the inner ear 16. These fluid waves stimulate several nerve endings 40 that, in turn, transmit sound energy to the brain where it is interpreted.

The ET 26 is a narrow, one-and-a-half-inch long channel connecting the middle ear 14 with the nasopharynx 30, the upper throat area just above the palate, in back of the nose. A narrowed region known as the isthmus 29 of the ET 26 provides a transition between the remainder of the ET 26 and the middle ear 14. The isthmus 29 is the narrowest part of the ET 26 at the junction of the bony and cartilaginous parts of the ET 26 (i.e., where the bony canal meets the cartilaginous tube). The isthmus 29 thus has a reduced inner diameter compared to the remaining portion of the ET 26 that extends between the isthmus 29 and the pharyngeal ostium 28; and provides a density that is substantially greater than the density of the tissue of the remaining portion of the ET 26 that extends between the isthmus 29 and the pharyngeal ostium 28.

The ET 26 functions as a pressure equalizing valve for the middle ear 14, which is normally filled with air. When functioning properly, the ET 26 opens for a fraction of a second periodically (about once every three minutes) in response to swallowing or yawning. In so doing, it allows air into the middle ear 14 to replace air that has been absorbed by the middle ear lining (mucous membrane) or to equalize pressure changes occurring on altitude changes. Anything that interferes with this periodic opening and closing of the ET 26 may result in hearing impairment or other ear symptoms.

In some cases, the ET 26 may fail to close properly, such that the ET 26 takes an inordinately prolonged amount of time to close after being opened, or such that the ET 26 substantially remains in a patulous state. This may adversely affect the patient by causing variations in the upper airway pressure around the ET 26 and the middle ear 14. In some patients, a patulous ET 26 may create a feeling of dry sinus, an increased breathing rate with physical activity, higher than usual perceived volumes of sound, and/or other undesirable consequences. It may therefore be desirable to provide a form of treatment for a patulous ET 26. It may further be desirable for such a treatment to still provide some degree of ventilation and drainage for the ET 26, without completely closing the ET 26.

While a variety of devices have been made and used to treat an ear of a patient, it is believed that no one prior to the inventors has made or used the inventions described in the appended claims.

### BRIEF DESCRIPTION OF THE DRAWINGS

The drawings and detailed description that follow are intended to be merely illustrative and are not intended to limit the scope of the inventions as contemplated by the inventors.

FIG. 1 depicts a cross-sectional view of a human ear showing the inner, middle, and outer ear portions and the Eustachian tube connecting the middle ear with the nasopharynx region of the throat, according to some embodiments.

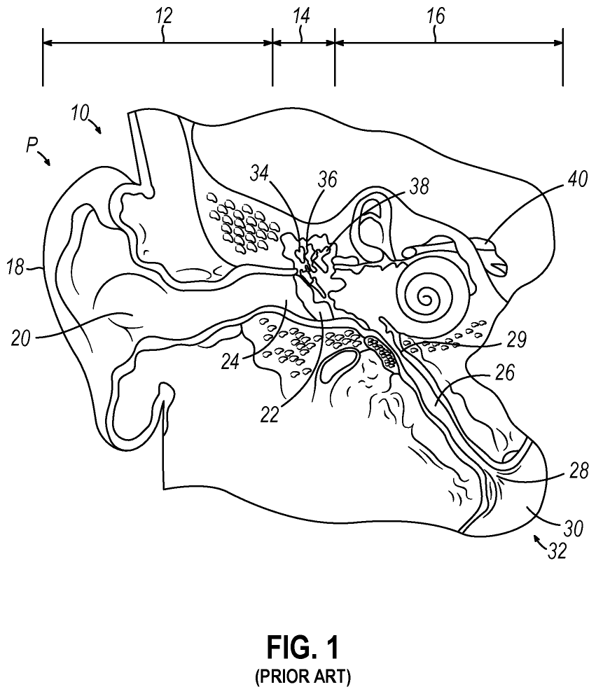

FIG. 2 depicts a side view of an example of an ET implant, according to some embodiments.

FIG. 3 depicts a cross-sectional side view of the ET implant of FIG. 2, according to some embodiments.

FIG. 4 depicts a side view of the ET implant of FIG. 2 loaded in a distal portion of a deployment instrument, according to some embodiments.

FIG. 5 depicts a cross-sectional view of the ET of FIG. 1, with the deployment instrument of FIG. 4 and ET implant of FIG. 2 being advanced toward the ET, according to some embodiments.

FIG. 6 depicts a cross-sectional view of the ET of FIG. 1 with a portion of the deployment instrument of FIG. 4 advancing the ET implant of FIG. 2 into the ET, where the ET implant is in a longitudinally extended, radially contracted configuration, according to some embodiments.

FIG. 7 depicts a cross-sectional view of the ET of FIG. 1 with a portion of the deployment instrument of FIG. 4 moving away from the ET implant of FIG. 2, where the ET implant is in a longitudinally contracted, radially expanded configuration, according to some embodiments.

FIG. 8 depicts a cross-sectional view of the ET implant of FIG. 2 implanted into the ET of FIG. 1, where the implant remains in the longitudinally contracted, radially expanded configuration, according to some embodiments.

### DETAILED DESCRIPTION

The following detailed description should be read with reference to the drawings, in which like elements in different drawings are identically numbered. The drawings, which are not necessarily to scale, depict exemplary examples for the purpose of explanation only and are not intended to limit the scope of the inventions. The detailed description illustrates by way of example, not by way of limitation, the principles of the inventions. This description will clearly enable one skilled in the art to make and use the inventions, and describes several examples, adaptations, variations, alternative and uses of the inventions, including what is presently believed to be the best mode of carrying out the inventions.

As used herein, the terms “about” and “approximately” for any numerical values or ranges indicate a suitable dimensional tolerance that allows the part or collection of components to function for its intended purpose as described herein.

### I. Example of Implant for Patulous ET

As noted above, some patients may have an ET 26 that remains patulous for a prolonged period, which may be undesirable for various reasons. In some instances, if a patulous ET 26 is maintained in a radially outward stressed state (as compared to an ET 26 in the patulous state) for a prolonged period of time (e.g. nine to twelve months), the prolonged stress may trigger a process were cells within the ET 26 regenerate such that the ET 26 transitions from the undesirable patulous state toward a more desirable, normal functioning state. For instance, imposing a radially outward stress on the ET 26 may generate scar tissue in the ET 26. It may therefore be desirable to insert an implant or other device into a patulous ET 26 of a patient, where the inserted implant or other device is capable of bearing radially outward against the sidewall of the patulous ET 26 for a prolonged period of time.

When an implant or other device is inserted and/or located within a patulous ET 26 of the patient for a prolonged period of time, the implant may cause discomfort or pain for a multitude of reasons, such as due to the rigidity of the implant along a longitudinal profile. Therefore, it may also be desirable to have an implant or other device that has sufficient flexibility to conform to the anatomical shape of a patulous ET 26 after or during deployment.

The following description provides an example of a device that may be deployed within the ET 26 to bear outwardly against the inner diameter of the ET 26 for a prolonged period of time. Other suitable ways in which the below-described device may be used will be apparent to those skilled in the art in view of the teachings herein.

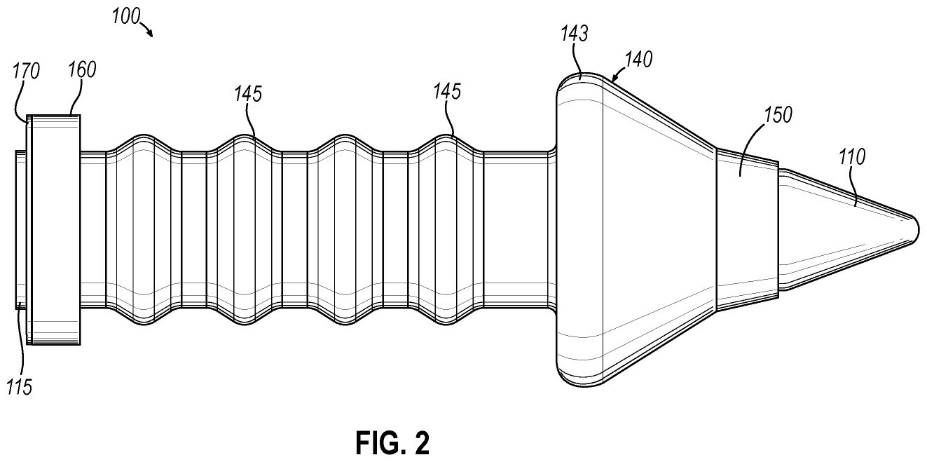

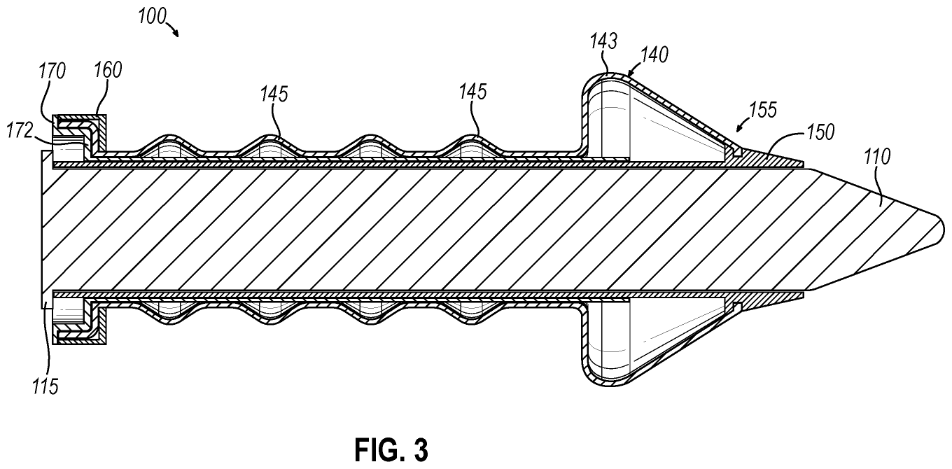

FIGS. 2 and 3 show an example implant 100 that may be deployed within the ET 26. The implant 100 includes a dilator 110, an elastomeric sleeve 140, an inner support 150, an outer retainer 160, and an outer support 170. The elastomeric sleeve 140 is positioned around a portion of the dilator 110, the inner support 150, and the outer support 170. The sleeve 140 includes a distal end and a proximal end. The proximal end of the sleeve 140 is positioned between and coupled to the outer support 170 and the outer retainer 160. The distal end of the sleeve 140 is coupled to a distal portion of the inner support 150. The sleeve 140 and the inner support 150 may be coupled via an overmold joint 155. Alternatively, any other suitable structure or technique may be used to couple the sleeve 140 with the inner support 150.

In the present example, the sleeve 140 includes a bulbous or pear-shaped portion 143 projecting radially around or raised above the inner and outer supports 150, 170. The pear-shaped portion 143 is configured to bear radially on the ET 26 when the implant 100 is positioned inside the ET 26. The sleeve 140 of the present example also includes a plurality of relief folds 145. Each relief fold 145 is positioned proximal to the pear-shaped portion 143 and includes a portion projecting radially outward from an adjacent remainder of the sleeve 140. The pear-shaped portion 143 and relief folds 145 define respective gaps between the sleeve 140 and the inner and outer supports 150, 170, as shown in FIG. 3.

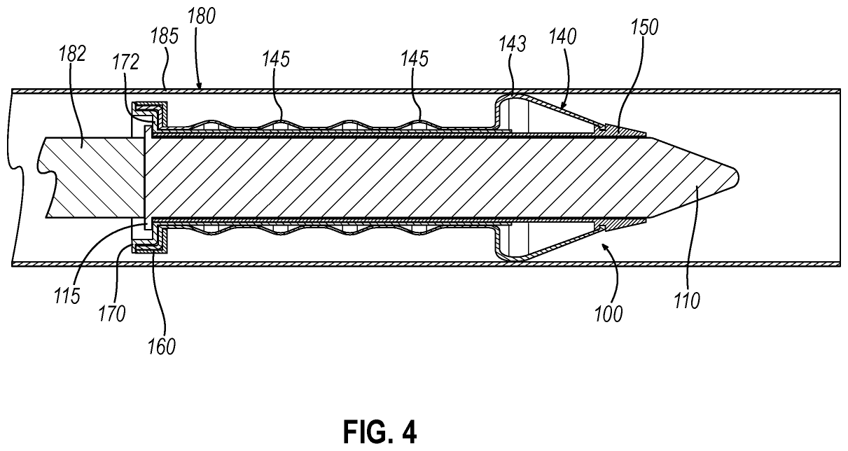

The sleeve 140 of the present example comprises an elastomeric material (e.g., silicone, etc.) and is resiliently biased toward a longitudinally contracted, radially expanded configuration such as that shown in FIG. 3. The elastomeric material forming the sleeve 140 is deformable radially inwardly and thus provides flexibility to conform to the anatomical shape of a patulous ET 26. In addition, as described below in the context of FIGS. 4-6, elastomeric material forming the sleeve 140 also allows the sleeve 140 to achieve a longitudinally extended, radially contracted configuration. The longitudinally extended, radially contracted configuration may be defined by when the flange 115 of the dilator 110 (described below) contacts the step 172 of the outer support 170 (also described below).

The outer support 170 radially supports a portion of the sleeve 140 and extends longitudinally from the pear-shaped portion 143 of the sleeve 140 to a proximal end of the implant 100. A proximal end of the outer support 170 includes a step 172. The step 172 is configured to restrain a proximal end of the sleeve 140, where the proximal end of the sleeve 140 is positioned or crimped between the step 172 and an outer retainer 160. As will be described in detail below, the step 172 may also be used to prevent the dilator 110 from translating distally beyond the step 172. The outer retainer 160 of the present example is shaped as an angled collar or crimp; and is sized to be larger than the ET 26 such as to not enter the ET 26 upon insertion of the implant 100.

The inner support 150 is positioned within the outer support 170. The inner support 150 is in the form of a sleeve or hollow cylinder in this example. A distal portion of the inner support 150 includes a tapered or angled portion that is sized and shaped to allow for smooth radial transition between the inner support 150 and the pear-shaped portion 143 of the sleeve 140 at the overmold joint 155. The inner support 150 extends proximally beyond a portion of the step 172 of the outer support 170.

The dilator 110 is positioned within the inner support 150. The dilator 110 is in the form of a cylinder or shaft in this example and includes a pointed (yet atraumatic) distal tip and a proximal flange 115. The pointed distal tip is sized and shaped to allow for a smooth radial transition between the dilator 110 and the distal portion of the inner support 150. The pointed distal tip includes a rounded distal-most end, such that the pointed distal tip is atraumatic to avoid piercing or scraping tissue during insertion into the ET 26. The flange 115 is positioned at a proximal most portion of the dilator 110 and projects radially relative to adjacent portions of the dilator 110 such that a proximal edge of the inner support 150 abuts a distal edge of the flange 115. The flange 115 extends radially outward relative to the inner support 150 such that the flange 115 may contact the step 172 of the outer support 170 and be prevented from translating beyond a proximal edge of the step 172. The inner support 150 may be press-fit or adhered to the dilator 110 such that the inner support 150 and the dilator 110 are configured to longitudinally translate together and relative to the outer retainer 160, the outer support 170, and the proximal portion of the sleeve 140. Optionally, the dilator 110 and/or remaining portions of the implant 100 may include one or more bores or channels (not shown) to provide ventilation and/or drainage of the ET 26 through the implant 100, but to a lesser degree than the ET 26 would otherwise vent/drain without the implant 100 being inserted.

### II. Example of Method of Treating a Patulous ET

FIGS. 4-8 show an example of a method of treating the ET with the implant 100. As shown in FIG. 4, the implant 100 may be loaded into an instrument 180. The instrument 180 of this example includes a sheath 185 and a rod 182, which is operable to translate relative to the sheath 185. The sheath 185 is sized to contain the implant 100 and provide some degree of radial compression of the sleeve 140 of the implant 100. The rod 182 is operable to bear distally against the implant 100 and thereby push the implant 100 distally out of the sheath 185 as described below. While not shown, the proximal end of the instrument 180 may include a handle or grip with an actuator that is operable to drive longitudinal movement of the rod 182 relative to the sheath 185. While the implant 100 is contained in the sheath 185 as shown in FIG. 4, the rod 182 bears distally against the proximal end of the dilator 110, and the friction between the sleeve 140 and the inner diameter of the sheath 185 substantially retains the implant 100 within the sheath 185. In addition, this arrangement provides the sleeve 140 in the longitudinally extended, radially contracted configuration while the implant 100 is disposed within the sheath 185.

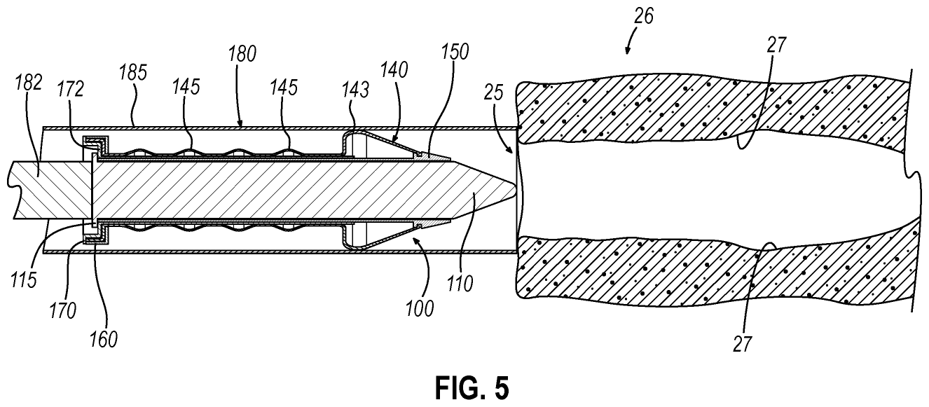

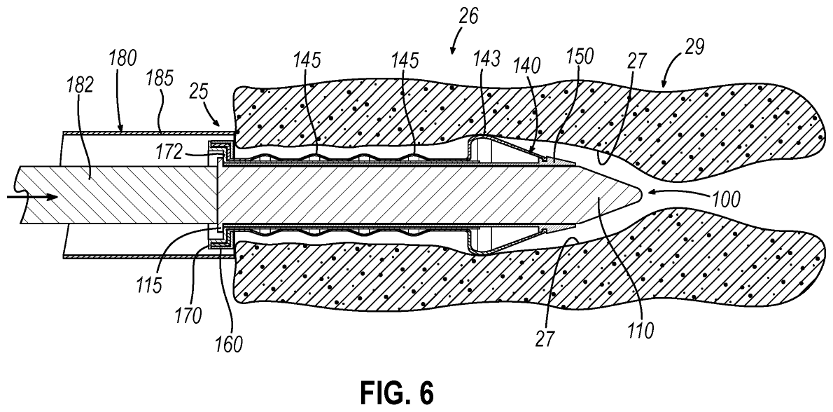

As shown in FIG. 5, an operator may position the instrument 180 such that the distal end of the sheath 185 abuts the pharyngeal ostium 28 of the ET 26, with the implant 100 being coaxially aligned with the ET 26 in preparation for insertion of the implant 100 into the ET 26. Once the instrument 180 and the implant 100 have been sufficiently positioned in relation to the ET 26, the operator may advance the rod 182 distally relative to the sheath 185 and thus apply a distally oriented force onto the flange 115, while holding the sheath 185 stationary relative to the ET 26. This distal translation of the rod 182 relative to the sheath 185 pushes the implant 100 distally out from the sheath 185 and into the ET 26, as shown in FIG. 6. The sleeve 140 remains in the longitudinally extended, radially contracted configuration as the rod 182 pushes the implant 100 distally out from the sheath 185 and into the ET 26. It should be understood that the ET 26 provides friction against the sleeve 140 as the implant 100 is being pushed distally into the ET 26, thereby allowing the dilator 110 to maintain a distal position in relation to the sleeve 140, overcoming the resilient bias of the sleeve 140 toward the longitudinally contracted, radially expanded configuration during the act of advancing the implant 100 into the ET 26. Moreover, the tapered shapes of the distal tip of the dilator 110 and the distal portion of the inner support 150 facilitate advancement of the implant 100 into the ET 26.

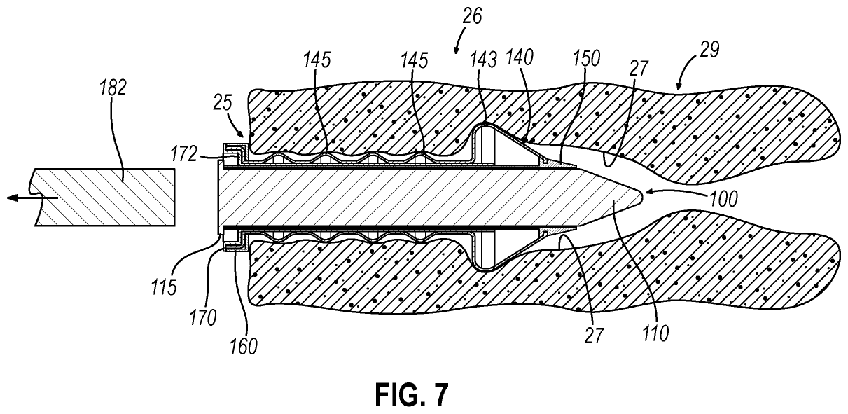

In the present example, due to the elastomeric nature of the material forming the sleeve 140, the pear-shaped portion 143 defines a first maximum diameter when the sleeve 140 is in the longitudinally extended, radially contracted configuration. When the sleeve 140 is in the longitudinally contracted, radially expanded configuration, the pear-shaped portion 143 defines a second maximum diameter that is larger than the first maximum diameter. When the sleeve 140 in the is in the longitudinally extended, radially contracted configuration (FIGS. 4-6), outer portions of the pear-shaped portion 143 and outer portions of the relief folds 145 may be positioned closer to the central longitudinal axis of the implant 100 than when the sleeve 140 is longitudinally contracted, radially expanded configuration (FIG. 7).

The implant 100 may be advanced distally into the ET 26 until the outer retainer 160 contacts the pharyngeal ostium 28 of the ET 26. Alternatively, the implant 100 may be advanced distally into the ET 26 to a point where the outer retainer 160 is positioned entirely in the ET. In the present example, the rod 182 continues to bear distally against the proximal end of the dilator 110, and the flange 115 remains in contact with the step 172, as the implant 100 is advanced distally into the ET 26. The sleeve 140 thus remains in the longitudinally extended, radially contracted configuration as the implant 100 is advanced distally into the ET 26. Once the implant 100 has been advanced to the desired depth within the ET 26, the rod 182 is retracted proximally as shown in FIG. 7 and the instrument 180 is removed from the patient.

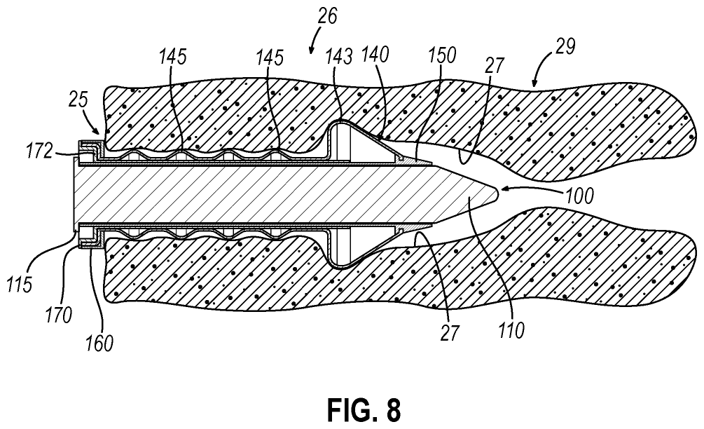

As shown in FIG. 8 with the instrument 180 completely removed from patient, when no distal force is being applied to the proximal end of the dilator 110 by the rod 182, the sleeve 140 resiliently transitions to the longitudinally contracted, radially expanded configuration, as described above. Once the sleeve 140 has transitioned to the longitudinally contracted, radially expanded configuration, the pear-shaped portion 143 and the relief folds 145 increase their contact with the sidewall of the ET 26, thereby resiliently bearing radially outward against the sidewall of the ET 26. This engagement of the pear-shaped portion 143 and the relief folds 145 with the sidewall of the ET 26 may provide sufficient friction to retain the implant 100 within the ET 26. In addition, engagement of the pear-shaped portion 143 and the relief folds 145 with the sidewall of the ET 26 may provide a fluid seal between opposing ends of the ET 26, such that the implanted the implant 100 may effectively obstruct an otherwise patulous ET 26.

As noted above, some variations of the implant 100 may provide one or more pathways for ventilation and/or fluid drainage through the ET 26, though such pathways may still provide a substantial reduction in ventilation through the ET 26 that would otherwise occur with a patulous ET 26 in the absence of the implant 100. In some such versions, the implant 100 may remain installed in the ET 26 indefinitely. In some other versions, the presence of the implant 100 within the ET 26 for a sufficient period of time may eventually lead to the formation of scar tissue in the ET 26 or otherwise effectively strengthen the ET 26. In some such scenarios, the implant 100 may be removed or bioabsorbed, and the post-implant ET 26 may effectively function as a non-patulous ET (ET) due to the scar tissue or other strengthening effects that were caused by the presence of the implant 100 in the ET for the sufficient period of time.

### III. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

Example 1: An implant comprising: (a) a shaft defining a longitudinal axis; and (b) an extendable sleeve including a proximal end and a distal end, the extendable sleeve being positioned around the shaft and configured to bear radially outward against a Eustachian tube of a patient, the distal end of the extendable sleeve being configured to translate with the shaft, the proximal end of the sleeve being configured to translate relative to the shaft.

Example 2: The implant of Example 1, a distal portion of the extendable sleeve including a pear-shaped portion configured to bear radially outward against the Eustachian tube of the patient.

Example 3: The implant of any one of the preceding Examples, the implant including an outer retainer at the distal end of the extendable sleeve.

Example 4: The implant of any one of the preceding Examples, the implant including an inner support positioned between and coupled to both the extendable sleeve and the shaft.

Example 5: The implant of Example 4, wherein the distal end of the extendable sleeve comprises an overmold secured to the inner support.

Example 6: The implant of any one of Examples 4 and 5, the inner support being configured to translate with the shaft and the distal end of the extendable sleeve.

Example 7: The implant of any one of the preceding Examples, the implant including an outer support positioned between the extendable sleeve and the shaft, the outer support being configured to translate relative to the shaft and the distal end of the extendable sleeve, the outer support being coupled with the proximal end of the extendable sleeve.

Example 8: The implant of Example 7, the outer support including a stop at a distal portion of the outer support.

Example 9: The implant of Example 8, the shaft including a flange at a proximal end of the shaft, the flange being configured to contact the stop to thereby prevent distal translation of the shaft beyond the stop.

Example 10: The implant of any one of the preceding Examples, the extendable sleeve further including a relief fold at a proximal section of the extendable sleeve, the relief fold being configured to transition between a first relief diameter and a second relief diameter.

Example 11: The implant of Example 10, wherein the relief fold being configured to bear radially outward against the Eustachian tube of the patient.

Example 12: The implant of any one of the preceding Examples, the extendable sleeve being configured to transition between a longitudinally contracted, radially expanded configuration and a longitudinally extended, radially contracted configuration, the extendable sleeve having a first maximum diameter in the longitudinally contracted, radially expanded configuration and a second maximum diameter in the longitudinally extended, radially contracted configuration, the first diameter being greater than the second diameter.

Example 13: The implant of Example 12, the shaft including a longitudinal axis, the shaft being configured to translate along the longitudinal axis and relative to the proximal end of the extendable sleeve to thereby transition the implant between the longitudinally contracted, radially expanded configuration and the longitudinally extended, radially contracted configuration.

Example 14: The implant of any one of Examples 12 and 13, the first maximum diameter being sized to plug the Eustachian tube of the patient, the second maximum diameter being sized to translate through a portion of the Eustachian tube of the patient.

Example 15: The implant of any one of the preceding Examples, the extendable sleeve including silicone.

Example 16: The implant of any one of the preceding Examples, the shaft including a distal end having a pointed tip.

Example 17: The implant of any one of the preceding Examples, each of the shaft and the extendable sleeve including taper angles that are each less than 90 degrees relative to the longitudinal axis.

Example 18: An apparatus, the apparatus including: (a) an implant configured to bear radially outward against a Eustachian tube of a patient and further configured to transition between a longitudinally contracted, radially expanded configuration and a longitudinally extended, radially contracted configuration, the implant comprising: (i) a shaft having a proximal end, and (ii) an extendable sleeve positioned around the shaft and being configured to resiliently bias the implant toward the longitudinally contracted, radially expanded configuration, the sleeve being configured to contact the Eustachian tube; (b) in instrument including a rod configured to apply a distal force to the proximal end of the shaft to thereby transition the implant from the longitudinally contracted, radially expanded configuration to the a longitudinally extended, radially contracted configuration when the extendable sleeve is in contact with the Eustachian tube.

Example 19: The apparatus of Example 18, the rod being operable to drive the implant distally into the Eustachian tube of the patient.

Example 20: The apparatus of any one of Examples 18 and 19, the implant including an outer retainer at a proximal portion of the implant, the outer retainer being having a larger diameter than remaining portions of the implant when the implant is in the longitudinally extended, radially contracted configuration, the outer retainer being configured to contact an opening of the Eustachian tube.

Example 21: A method of treating a patulous Eustachian tube, the method comprising: (a) inserting an implant into the Eustachian tube while the implant is in a longitudinally extended, radially contracted configuration, the implant having an inner component at a first longitudinal position relative to an outer component during the act of inserting, the positioning of the inner component at the first longitudinal position relative to the outer component providing the longitudinally extended, radially contracted configuration to the implant; and (b) releasing the inserted implant within the Eustachian tube, the inner component transitioning to a second longitudinal position relative to the outer component after release of the inserted implant within the Eustachian tube, the outer component resiliently biasing the implant toward a longitudinally contracted, radially expanded configuration after release of the inserted implant within the Eustachian tube.

Example 22: A method of reducing a maximum diameter of an implant configured to be inserted into a Eustachian tube of a patient, the implant including a shaft and an extendable sleeve, the extendable sleeve including a proximal end, a distal end, and a maximum diameter portion, the extendable sleeve being positioned around the shaft, the method including translating the shaft distally relative to the proximal end of the extendable sleeve, to thereby translate the distal end of the extendable sleeve relative to the proximal end of the extendable sleeve, to thereby reduce an outer diameter defined by the maximum diameter portion.

### IV. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, examples, examples, etc. described herein may be combined with any one or more of the other teachings, expressions, examples, examples, etc. that are described herein. The above-described teachings, expressions, examples, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various examples of the present inventions, further adaptations of the methods and systems described herein may be accomplished by appropriate modifications by one of ordinary skill in the art without departing from the scope of the present inventions. Several of such potential modifications have been mentioned, and others will be apparent to those skilled in the art. For instance, the examples, geometrics, materials, dimensions, ratios, steps, and the like discussed above are illustrative and are not required. Accordingly, the scope of the present inventions should be considered in terms of the following claims and is understood not to be limited to the details of structure and operation shown and described in the specification and drawings.

What is claimed is:

1. An implant comprising:
a shaft defining a longitudinal axis; and
an extendable sleeve comprising a proximal end and a distal end, wherein the extendable sleeve is positioned around the shaft and configured to bear radially outward against a Eustachian tube of a patient, the proximal end of the sleeve is configured to translate relative to the shaft, and the distal end of the extendable sleeve is configured to translate with the shaft.

2. The implant of claim 1, wherein a distal portion of the extendable sleeve comprises a pear-shaped portion configured to bear radially outward against the Eustachian tube of the patient.

3. The implant of claim 1, wherein the implant comprises an outer retainer at the distal end of the extendable sleeve.

4. The implant of claim 1, wherein the implant comprises an inner support positioned between and coupled to both the extendable sleeve and the shaft, and the implant is configured to translate with the shaft and the distal end of the extendable sleeve.

5. The implant of claim 4, wherein the distal end of the extendable sleeve comprises an overmold secured to the inner support.

6. The implant of claim 1, wherein the implant comprises an outer support positioned between the extendable sleeve and the shaft, and the outer support is coupled with the proximal end of the extendable sleeve and configured to translate relative to the shaft and the distal end of the extendable sleeve.

7. The implant of claim 6, wherein the outer support comprises a stop at a distal portion of the outer support.

8. The implant of claim 7, wherein the shaft comprises a flange at a proximal end of the shaft, wherein the flange is configured to contact the stop, thereby preventing distal translation of the shaft beyond the stop.

9. The implant of claim 1, wherein the extendable sleeve comprises a relief fold at a proximal section of the extendable sleeve, the relief fold being configured to transition between a first relief diameter and a second relief diameter and to bear radially outward against the Eustachian tube of the patient.

10. The implant of claim 1, wherein the extendable sleeve is configured to transition between a longitudinally contracted, radially expanded configuration and a longitudinally extended, radially contracted configuration, the extendable sleeve having a first maximum diameter in the longitudinally contracted, radially expanded configuration and a second maximum diameter in the longitudinally extended, radially contracted configuration, the first maximum diameter being greater than the second maximum diameter.

11. The implant of claim 10, wherein the shaft comprises a longitudinal axis and is configured to translate along the longitudinal axis and relative to the proximal end of the extendable sleeve, thereby transitioning the implant between the longitudinally contracted, radially expanded configuration and the longitudinally extended, radially contracted configuration.

12. The implant of claim 11, wherein the first maximum diameter is sized to plug the Eustachian tube of the patient, and the second maximum diameter is sized to translate through a portion of the Eustachian tube of the patient.

13. The implant of claim 1, wherein the extendable sleeve comprises silicone.

14. The implant of claim 1, wherein the shaft comprises a distal end having a pointed tip.

15. The implant of claim 1, wherein the shaft and the extendable sleeve each comprise a taper angle that is 90 degrees or less relative to the longitudinal axis.

16. An apparatus, the apparatus comprising:
an implant configured to bear radially outward against a Eustachian tube of a patient and to transition between a longitudinally contracted, radially expanded configuration and a longitudinally extended, radially contracted configuration, the implant comprising:
a shaft having a proximal end, and
an extendable sleeve positioned around the shaft and configured to resiliently bias the implant toward the longitudinally contracted, radially expanded configuration, the extendable sleeve being configured to contact the Eustachian tube; and
an instrument comprising a rod configured to apply a distal force to the proximal end of the shaft to thereby transition the implant from the longitudinally contracted, radially expanded configuration to the longitudinally extended, radially contracted configuration when the extendable sleeve is in contact with the Eustachian tube.

17. The apparatus of claim 16, wherein the rod is operable to drive the implant distally into the Eustachian tube of the patient.

18. The apparatus of claim 17, wherein the implant comprises an outer retainer at a proximal portion of the implant, the outer retainer having a larger diameter than remaining portions of the implant when the implant is in the longitudinally extended, radially contracted configuration and being configured to contact an opening of the Eustachian tube.

19. A method of treating a patulous Eustachian tube, the method comprising:
inserting an implant into the Eustachian tube while the implant is in a longitudinally extended, radially contracted configuration, wherein the implant is in the longitudinally extended, radially contracted configuration when an inner component of the implant is at a first longitudinal position relative to an outer component of the implant; and
releasing the inserted implant within the Eustachian tube such that the inner component transitions to a second longitudinal position relative to the outer component upon release, wherein the outer component resiliently biases the implant toward a longitudinally contracted, radially expanded configuration after release.
